(19) United States
(12) Patent Application Publication
(10) Pub. No.: US 2018/0304090 A1
(43) Pub. Date: Oct. 25, 2018

(54) PHOTOELECTRIC RESONANCE CHIP AND METHOD FOR CONDUCTING MICROWAVES THROUGH THE SAME

(71) Applicants: JIN-SHENG TSENG, NEW TAIPEI CITY (TW); JING-WEN LIAO, NEW TAIPEI CITY (TW); SHU-HUA SHIAO, NEW TAIPEI CITY (TW)

(72) Inventors: JIN-SHENG TSENG, NEW TAIPEI CITY (TW); JING-WEN LIAO, NEW TAIPEI CITY (TW); SHU-HUA SHIAO, NEW TAIPEI CITY (TW)

(21) Appl. No.: 16/018,064

(22) Filed: Jun. 26, 2018

Related U.S. Application Data
(63) Continuation-in-part of application No. 15/157,413, filed on May 18, 2016.

(30) Foreign Application Priority Data
Jan. 15, 2016 (TW) 105200621

(51) Int. Cl. A61N 5/02; A61H 39/00; A61N 5/04

(52) U.S. Cl. CPC A61N 5/025; A61H 2039/005; A61N 5/04; A61H 39/00

(57) ABSTRACT

A photoelectric resonance chip includes, a chip main body, signal receiver having a first coil, encoding device, signal generator having a second coil; and at least one light wave generating element and at least one electric wave generating element. When a user carries the chip main body on their body, the first coil converts the type of external microwave from energy wave to current, the signal receiver receives the signals and transmits them to the encoding device, the encoding device increases the frequencies of the signals by means modulation to enlarge the power thereof and transmits them to the signal generator, the second coil converts the type of the signal from current to energy wave, the signal generator feeds the enlarged microwave back to the user's body, and the microwave fed back to the body carries light wave energy and electric wave energy to flow around the whole body.

### CROSS-REFERENCE TO RELATED APPLICATION

This is a continuation-in-part of co-pending U.S. patent application Ser. No. 15/157,413 filed on May 18, 2016 and owned by the present applicant.

### (a) TECHNICAL FIELD OF THE INVENTION

The present invention relates to a photoelectric resonance chip and a method for conducting microwaves through the same, using microwave conduction to achieve the object of feeding a local action back to a whole human body, while possessing the effects of light wave energy and electric wave energy.

### (b) DESCRIPTION OF THE PRIOR ART

Health patches are patches attached to a part of human body wanting to be improved and emitting far infrared through magnets therein to strengthen the circulation of a user's body, thereby improving the body's condition.

Such kinds of health patches have some problems in use:
1. magnets themselves are radioactive, being possibly dangerous to cause damage to a human body, and the radioactivity thereof will decay gradually in a long-term use, and finally, will disappear completely to lose the effect.
2. the energy cannot be concentrated effectively because the wavelength bandwidths of the magnets themselves are too broad such that the effect of improving human health is limited; although large machines such as far infrared generators whose bandwidths are rather concentrated are available in the market, they are not portable and incapable of being in prolonged contact with a human body to make far infrared effective such that they do not conform to modern people's requirements.
3. because the effective range of a conventional health patch is rather small and it can only effect locally in the vicinity thereof, more patches are required to attach one by one to a human body if many places on the body or the whole body need be conditioned.

### SUMMARY OF THE INVENTION

To overcome the defects of conventional health patches mentioned above, for example, lower repeated usage, necessary direct contact with a human body, rather local effect, and more complicated operation, the present invention is proposed.

One object of the present invention is to provide a photoelectric resonance chip and a method for conducting microwaves through the chip, utilizing actions such as absorption, strengthening and feeding-back to conduct microwaves to a human body, and adding light wave energy and electric wave energy in the microwaves to increase human body's bioelectrical energy, and at same time to apply the light wave energy and electric wave energy to the whole body with the microwaves.

To achieve the object mentioned above, the present invention proposes a photoelectric resonance chip and a method for conducting microwaves through the chip, where the photoelectric resonance chip includes: a chip main body; a signal receiver, configured inside the chip main body and having a first coil; an encoding device; a signal generator, having a second coil; and at least one light wave generating element and at least one electric wave generating element, configured on one side of the chip main body. When a user carries the chip main body on their body, the first coil of the signal receiver of the chip main body converts the type of external microwave from a energy wave to current; the signal receiver receives the signals and transmits them to the encoding device; the encoding device increases the frequencies of the signals by means modulation to enlarge the power thereof and transmits them to the signal generator; the second coil converts the type of signal from current to energy wave; the signal generator feeds the enlarged microwave back to the user's body; and the microwave fed back to the body carries light wave energy and electric wave energy to flow around the whole body, thereby strengthening body's biological waves and enhancing the efficacy of light wave energy and electric wave energy on a human body.

### BRIEF DESCRIPTION OF THE DRAWINGS

FIGS. 1 and 2 respectively are a perspective view of the present invention;

FIG. 3 is a flow chart of the present invention;

FIGS. 4, 5 and 6 respectively are a schematic view of the present invention upon implementation; and

FIG. 7 is a schematic view of another preferred embodiment of the present invention.

### DETAILED DESCRIPTION OF THE PREFERRED EMBODIMENTS

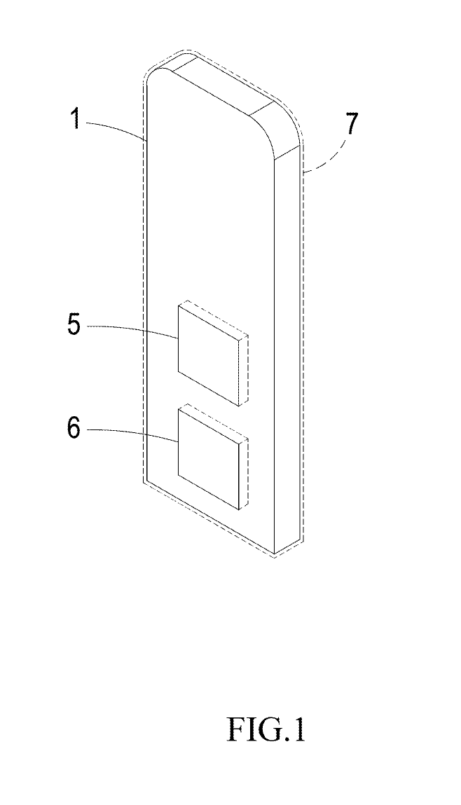

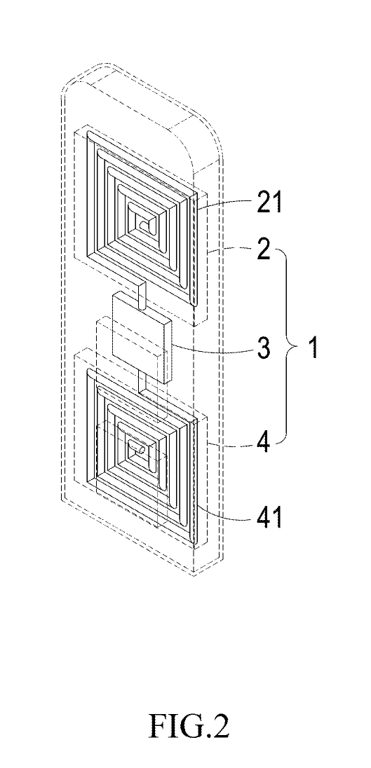

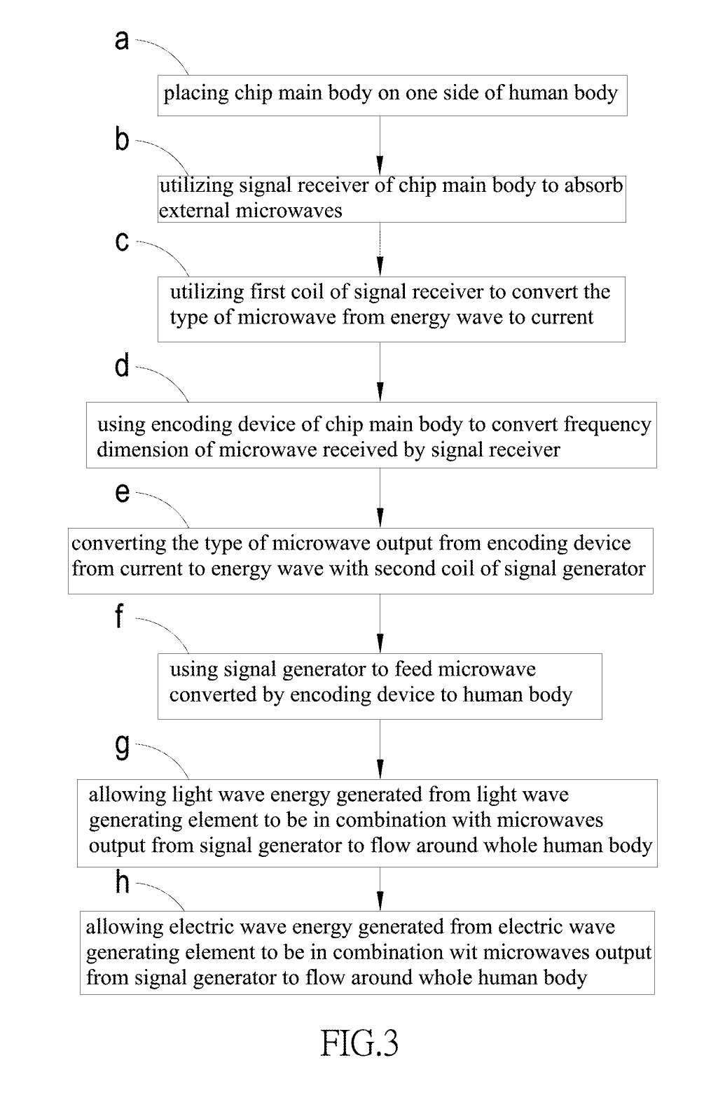

Referring to FIGS. 1 to 3, a photoelectric resonance chip according to the present invention includes a chip main body 1; a signal receiver 2, configured inside the chip main body 1 for the absorption of external microwave, and including at least one coil 21 adapted to convert the type of the microwave between energy wave and current; an encoding device, configured inside the chip main body 1, in electric connection with the signal receiver 2, and adapted to convert the frequency dimension of the microwave received by the signal receiver 2; a signal generator 4, configured inside the chip main body 1, in electric connection with the encoding device, adapted to feed the microwave output from the encoding device 3 back to a human body, and including at least one second coil 41 adapted to covert the type of the microwave between current and energy wave, the microwaves ranged from 0.01 milliamperes (mA) to 10 milliamperes (mA); at least one light wave generating element 5, configured on one side of the chip main body 1 and used to generate light wave energy capable of being in combination with the microwaves feeding back to a human body; at least one electric wave generating element 6, configured on one side of the chip main body 1, and used to generate electric wave energy capable of being in combination with the microwaves feeding back to a human body; and at least one protective shield 7, covering the chip main body 1 and made of flexible material, rigid material or nanometer material.

A method for conducting microwaves through a photoelectric resonance chip according to the present invention includes the steps as following:

(a) placing the chip main body on one side of a human body;

(b) using the signal receiver of the chip main body to absorb external microwaves;

(c) converting the type of the microwave from energy wave to current with the first coil of the signal receiver;

(d) converting the frequency dimension of the microwave received by the signal receiver with the encoding device of the chip main body;

(e) converting the type of the microwave output from the encoding device from current to energy wave with the second coil of the signal generator;

(f) feeding the microwave converted by the encoding device back to the human body with the signal generator;

(g) allowing light wave energy generated from the light wave generating element to be in combination with the microwaves output from the signal generator to flow around the whole human body; and

(h) allowing electric wave energy generated from the electric wave generating element to be in combination with the microwaves output from the signal generator to flow around the whole human body.

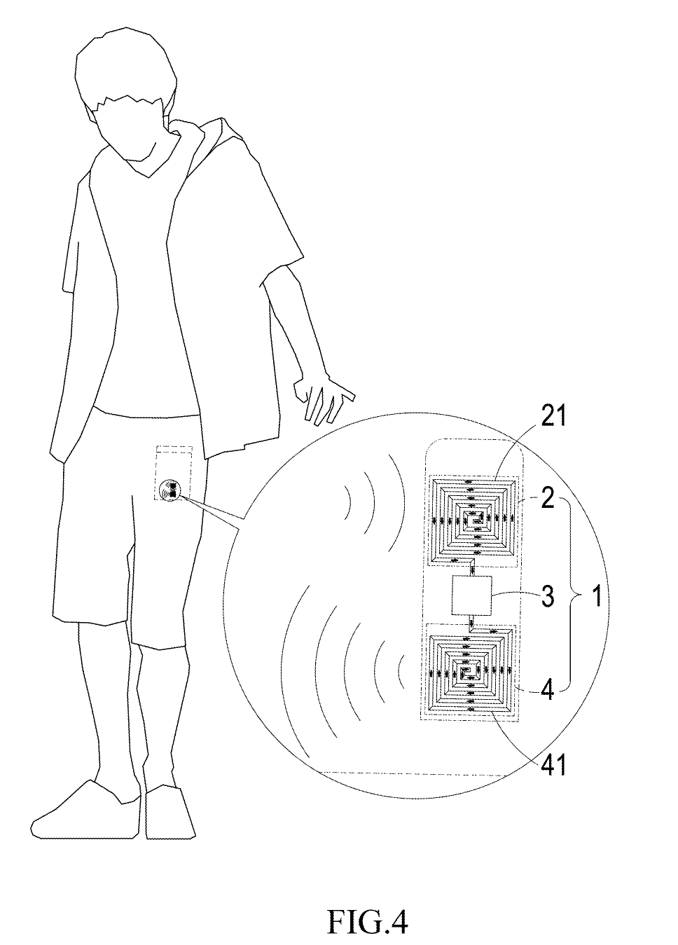

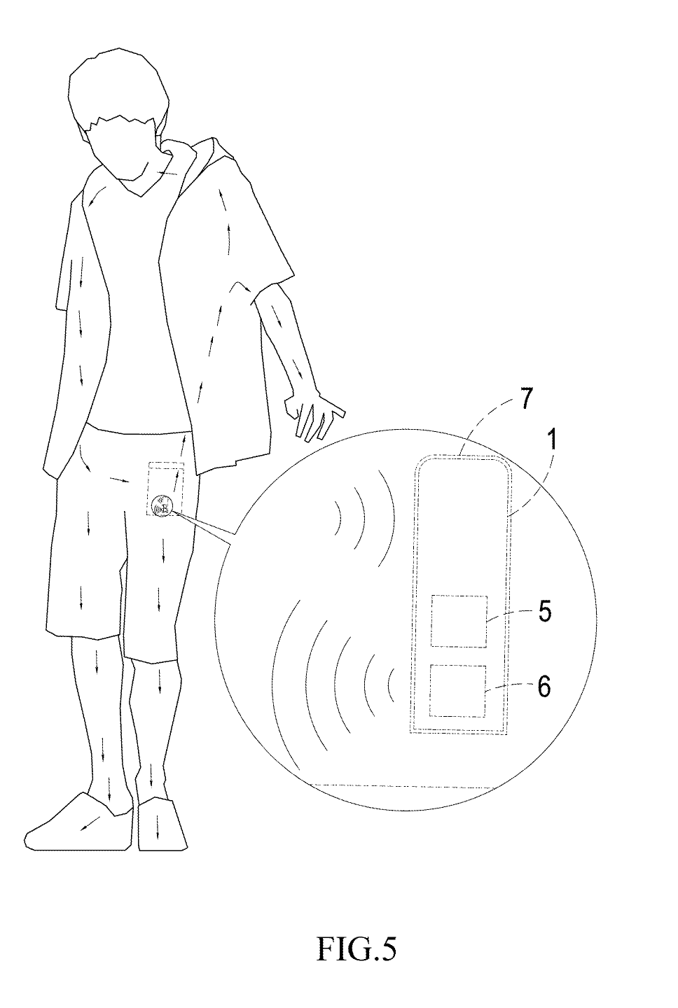

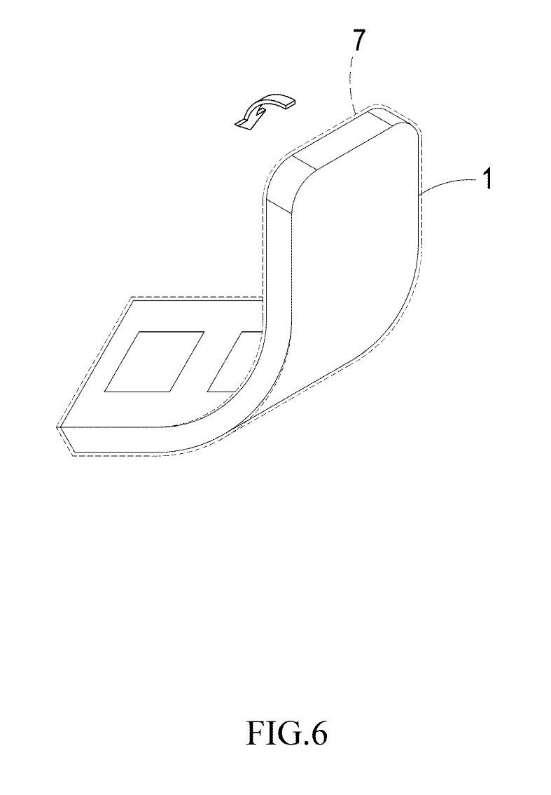

The present invention, as FIGS. 1 to 6 show, is a chip structure which is simply structured, lightweight and easy to carry; the area thereof approximately is smaller than 25 cm². In one preferred embodiment, the chip is a small area body which is 4 cm in length and 2 cm in width suitable for a user to carry and occupying no space. Furthermore, because the microwave radiation energy generated from the chip main body is very low, approximately one-thousandth of a Bluetooth earphone (currently, the safety standard for mobile phones announced by the US Federal Communication Committee is 1.6 w/kg, the safety standard of Europe is 2.0 w/kg recommended by World Health Organization, and the radiation energy of a Bluetooth earphone only is one-thirtieth of the standard value), it has no negative impact to a human body.

In actual use, the effective power range of the chip main body 1 is approximately ranged from 1 cm to 4 cm in order to allow the energy thereof to be absorbed by a human body because the final output frequency thereof falls in the range between 300 MHz to 30 Hz of ultrahigh frequency. Therefore, a user can use the chip of the present invention only by placing it in a pocket or combining it with a wearing object such as clothes, a hat or a necklace, and the chip can then be close to a human body's surface naturally. In this situation, the first coil 21 of the signal receiver 2 is used to receive external microwave including environmental microwave and weak electric wave in a human body to covert the microwave of an energy type to the one of a current type by means of electromagnetic induction, and the inducted current is transmitted from the signal receiver 2 to the encoding device 3. At this time, the encoding device then uses modulation to convert the low frequency wireless electromagnetic wave data of the inducted current to high frequency wireless electromagnetic signals, and electrons in the inducted current will jump from one stationary state to another so that radiation intensity will thus be increased. Next, the inducted current is then transmitted to the signal generator 4, allowing the second coir 41 of the signal generator 4 to convert the microwave of a current type to the one of an energy type by means of electromagnetic effect, and at the same time, energy is emitted in the form of electromagnetic waves.

Therefore, it is unnecessary to attach the chip main body 1 close to the human body and the absorption action can then be carried out, the power can be enlarged and the microwaves with the enlarged power are fed back to the human body. Furthermore, the microwaves are allowed to carry the light wave energy and electric wave energy generated respectively by the light wave generating element 5 and electric wave generating element 6 (in the embodiment, the light wave generating element 5 is an infrared light generator, and the electric wave generating element 6 is tourmaline (for the generation of positive ions) or negative ion generating device (for generation of negative ions)) by means of co-frequency resonance in such a way as to allow the microwaves themselves together with the light wave energy and electric wave energy acted locally to flow around the whole human body. Furthermore, the microwaves can generate toroidal magnetic fields which can speed blood flow; the formation of the toroidal magnetic fields is similar to adding many pipeline booster pumps in a blood system; the toroidal magnetic fields so generated have not only a thrusting action to speed blood but a moving action to move all cells in main and collateral channels such that they can strengthen bioelectrical and speed cell repair. As a result, a user can improve the circulation condition adjacent to the chip and achieve the effects of improving the human body's health, for example, strengthening the human body's main and collateral channels and acupuncture points, improving blood flow speed and volume inside blood vessels through the microwaves emitted from the user themselves and the cyclic operation mentioned above. When the health condition of a human body is improved, the microwaves emitted therefrom will be strengthened gradually, and the circulation will be more strengthened by means of the method mentioned above in such a way as to increase local temperature of the human body, and further to achieve the efficiencies of activating internal organs, enhancing immunity, increasing basal metabolic rate, improving blood circulation and adjusting the autonomic nerves, the function of hormonal balance and other effects. Thereupon, the human body health condition will further be improved with diet control and moderate exercise.

Furthermore, since the volume of the chip of the present invention is small, if the point of a human body where it is placed is close to a specific acupuncture point, not only a systemic effect can be generated through the microwaves flowing around the whole body, but the efficiency of acupuncture point stimulation can be brought into full play. The Acupuncture points are sensitive and functional points on main and collateral channels and contacts of bioelectrical, and main and collateral channels are the channels for the transmission of bioelectrical. An electric voltage and potential will be changed when a magnetic field is applied on an acupuncture point to excite bioelectrical to generate electromagnetic waves, and the generated electromagnetic waves are transmitted to the main and collateral channels of the whole body to form stimulation to the central nerve when they are transmitted thereto, thereby adjusting a lesion or lesions.

The co-frequency resonance principle mentioned above is utilizing the special physical function of co-frequency oscillation, allowing light particles to impact the molecules of the molecular structure of all organisms at a light speed without utilizing any media, strengthening the molecular structure to generate a vibration action immediately, and achieving the activation of the molecules. If it is explained with quantum mechanics, the phenomenon mentioned above is called photoelectric effect. According to the known basic scientific theories, all organisms (including human body) are constituted by molecules formed by the gathering of atoms and having their own specific vibration frequencies. Thereupon, the vibration frequency of a human body is interacted synchronously with co-frequency resonance energy, allowing the energy of the body to be increased. In addition, the chip body 1 absorbing external microwaves and electromagnetic waves can decrease the interference of the electromagnetic field generated from computer, mobile phone, or the like to a human body. Whereby, the present invention can strengthen a user's physical strength, endurance and resistance to stress, as well as, for example, improve the performance of the mind and focus attention.

Furthermore, high body temperature (36.5° C. to 37° C.) caused by the chip inside a human body facilitates the stronger mobility of enzymes; body temperature increases by 1 degree Celsius, the number of pulses per minute will increase by 10, resulting in the body being frill of fresh oxygen, cell metabolism being also increased. In addition, high body temperature may also make blood vessels become softer such that blood can be transmitted to the whole body more smoothly; if the temperature of a human body can be kept uniform, the autonomic nervous and hormonal secretion will also be maintained balanced, and the normal functions thereof can be brought into full play. Thereupon, the present invention allowing local temperature to be increased can facilitate a human body's health.

Because the encoding device 3 is a passive element, the whole chip receives microwaves emitted from a human body or existing in the environment in a passive way such that there is no radioactive decay and no radioactivity to hurt the human body. In addition, the action way of receiving microwaves passively allows the chip to be used forever without an electricity supply problem. Furthermore, the chip main body 1 is prevented from being exposed directly to and damaged by the atmosphere because the protective shield 4 carries out the protection, thereby allowing the whole chip to be more durable, small-sized. Since the protective shield 4 is made of nanometer material, the chip main body 1 may have certain flexibility such that it is not affect by deformation such as compression or curling while being placed inside some pocket or clothes and can be used normally.

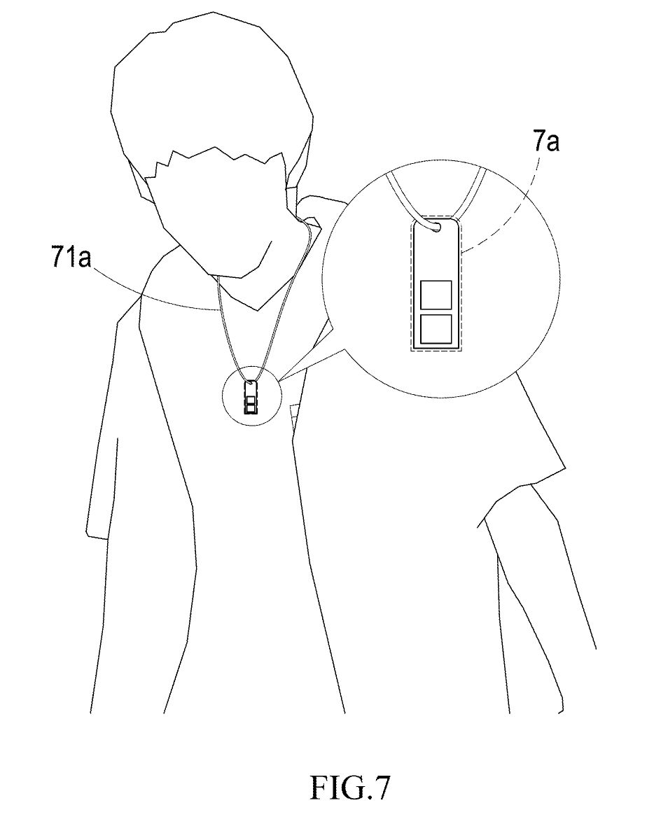

Referring to FIG. 7, the protective shield 7a is made of rigid material, on which a fixing element 71a is configured additionally, where the fixing element 71a is adapted to fix to a human body. Specifically, the fixing element 71a may be a rope or strap which is replaceable depending on use requirements; the fixing element 71a, shown in the figure, is a longer rope, and the protective shield 4a is modeled, allowing more flexible use collocation thereof.

I claim:

1. A photoelectric resonance chip, comprising:
a chip main body;
a signal receiver, configured inside said chip main body, adapted to receive external microwave, and comprising at least one first coil adapted to convert the type of said microwave between energy wave and current;
an encoding device, configured inside said chip main body, in electric connection with said signal receiver, and adapted to convert a frequency dimension of said microwave received by said signal receiver;
a signal generator, configured inside said chip main body, in electric connection with said encoding device, feeding said microwave output from said encoding device back to a human body, and comprising at least one second coil adapted to convert the type of said microwave wave output from said encoding device between current and energy wave;
at least one light wave generating element, configured on one side of said chip main body and adapted to generate light wave energy capable of being in combination with said microwaves fed back to said human body; and
at least one electric wave generating element, configured on one side of said chip main body and adapted to generate electric wave energy capable of being in combination with said microwaves fed back to said human body.

2. The chip according to claim 1, further comprising at least one protective shield covering said chip main body, and said protective shield is made of flexible material, rigid material or nanometer material.

3. The chip according claim 2, wherein said protective shield has at least one fixing element for the fixation to a human body.

4. The chip according to claim 1, wherein said chip main body is flexible.

5. The chip according to claim 1, wherein said microwaves are ranged from 0.01 milliamperes (mA) to 10 milliamperes (mA).

6. A method for conducting microwaves through a photoelectric resonance chip, comprising the following steps:
(a) placing a chip main body on one side of a human body;
(b) utilizing a signal receiver of said chip main body to absorb external microwaves;
(c) converting the type of said microwave from energy wave to current with a first coil of said signal receiver;
(d) converting the frequency dimension of said microwave received by said signal receiver with an encoding device of said chip main body;
(e) converting the type of said microwave output from said encoding device from current to energy wave with a second coil of a signal generator;
(f) feeding said microwave converted by said encoding device back to a human body with said signal generator;
(g) allowing light wave energy generated from a light wave generating element to be in combination with said microwaves output from said signal generator to flow around said whole human body; and
(h) allowing electric wave energy generated from an electric wave generating element to be in combination with said microwaves output from said signal generator to flow around said whole human body.

7. The method according to claim 6, wherein said photoelectric resonance chip further comprises at least one protective shield covering said chip main body, and said protective shield is made of flexible material, rigid material or nanometer material.

8. The method according to claim 7, wherein said protective shield has at least one fixing element for the fixation to said human body.

9. The method according to claim 6, wherein said chip main body is flexible.

10. The method according to claim 6, wherein said microwaves are ranged from 0.01 milliamperes (mA) to 10 milliamperes (mA).
